SEQUENCE LISTING (157 sequences)

SEQ ID NO: 1 - RNA, 28 nt, Artificial Sequence
  h.C11orf95RELA fusion Guide RNA
gcccuugggcgggcaagcugggacaccg

SEQ ID NO: 2 - RNA, 28 nt, Artificial Sequence
  h.C11orf95RELA fusion Guide RNA
ugggcccuugggcgggcaagcugggaca

SEQ ID NO: 3 - RNA, 28 nt, Artificial Sequence
  h.C11orf95RELA fusion Guide RNA
uucugggcccuugggcgggcaagcuggg

SEQ ID NO: 4 - RNA, 28 nt, Artificial Sequence
  h.C11orf95RELA fusion Guide RNA
aguucugggcccuugggcgggcaagcug

SEQ ID NO: 5 - RNA, 28 nt, Artificial Sequence
  h.C11orf95RELA fusion Guide RNA
gggaacaguucugggcccuugggcgggc

SEQ ID NO: 6 - RNA, 28 nt, Artificial Sequence
  h.C11orf95RELA fusion Guide RNA
agggggaacaguucugggcccuugggcg

SEQ ID NO: 7 - RNA, 28 nt, Artificial Sequence
  h.C11orf95RELA fusion Guide RNA
augagggggaacaguucugggcccuugg

SEQ ID NO: 8 - RNA, 28 nt, Artificial Sequence
  h.C11orf95RELA fusion Guide RNA
aagaugagggggaacaguucugggcccu

SEQ ID NO: 9 - RNA, 28 nt, Artificial Sequence
  h.C11orf95RELA fusion Guide RNA
gggaagaugagggggaacaguucugggc

SEQ ID NO: 10 - RNA, 28 nt, Artificial Sequence
  h.C11orf95RELA fusion Guide RNA
aacaguucugggcccuugggcgggcaag

SEQ ID NO: 11 - RNA, 28 nt, Artificial Sequence
  SS18-SSX fusion Guide Guide RNA
gcaugaucuggucauauccauaaggccu

SEQ ID NO: 12 - RNA, 28 nt, Artificial Sequence
  SS18-SSX fusion Guide Guide RNA
uugggcaugaucuggucauauccauaag

SEQ ID NO: 13 - RNA, 28 nt, Artificial Sequence
  SS18-SSX fusion Guide Guide RNA
gcuucuugggcaugaucuggucauaucc

SEQ ID NO: 14 - RNA, 28 nt, Artificial Sequence
  SS18-SSX fusion Guide Guide RNA
cuggcuucuugggcaugaucuggucaua

SEQ ID NO: 15 - RNA, 28 nt, Artificial Sequence
  SS18-SSX fusion Guide Guide RNA
ccucugcuggcuucuugggcaugaucug

SEQ ID NO: 16 - DNA, 28 bp, Artificial Sequence
  Guides targeting the ss Regions in loops in the XIST transcript
agactaggggtttgctgggagcagggct

SEQ ID NO: 17 - DNA, 28 bp, Artificial Sequence
  Guides targeting the ss Regions in loops in the XIST transcript
agccctgctcccagcaaacccctagtct

SEQ ID NO: 18 - DNA, 28 bp, Artificial Sequence
  Guides targeting the ss Regions in loops in the XIST transcript
ggggctagactaggggtttgctgggagc

SEQ ID NO: 19 - DNA, 28 bp, Artificial Sequence
  Guides targeting the ss Regions in loops in the XIST transcript
gctcccagcaaacccctagtctagcccc

SEQ ID NO: 20 - DNA, 28 bp, Artificial Sequence
  Guides targeting the ss Regions in loops in the XIST transcript
ggggggttaggggactggggctggggca

SEQ ID NO: 21 - DNA, 28 bp, Artificial Sequence
  Guides targeting the ss Regions in loops in the XIST transcript
tgccccagccccagtcccctaacccccc

SEQ ID NO: 22 - DNA, 28 bp, Artificial Sequence
  Guides targeting the ss Regions in loops in the XIST transcript
gggactggggctagggctggggggttag

SEQ ID NO: 23 - DNA, 28 bp, Artificial Sequence
  Guides targeting the ss Regions in loops in the XIST transcript
ctaaccccccagccctagccccagtccc

SEQ ID NO: 24 - DNA, 28 bp, Artificial Sequence
  Guides targeting the ss Regions in loops in the XIST transcript
gctgggattacaggtgtgagccaccaca

SEQ ID NO: 25 - DNA, 28 bp, Artificial Sequence
  Guides targeting the ss Regions in loops in the XIST transcript
tgtggtggctcacacctgtaatcccagc

SEQ ID NO: 26 - DNA, 28 bp, Artificial Sequence
  Guides targeting the ss Regions in loops in the XIST transcript
cccaaagtgctgggattacaggtgtgag

SEQ ID NO: 27 - DNA, 28 bp, Artificial Sequence
  Guides targeting the ss Regions in loops in the XIST transcript
ctcacacctgtaatcccagcactttggg

SEQ ID NO: 28 - DNA, 28 bp, Artificial Sequence
  Guides targeting the ss Regions in loops in the XIST transcript
aagggatcttcccacctcagcctcccaa

SEQ ID NO: 29 - DNA, 28 bp, Artificial Sequence
  Guides targeting the ss Regions in loops in the XIST transcript
ttgggaggctgaggtgggaagatccctt

SEQ ID NO: 30 - DNA, 28 bp, Artificial Sequence
  Guides targeting the ss Regions in loops in the XIST transcript
cctggcagtaagggatcttcccacctca

SEQ ID NO: 31 - DNA, 28 bp, Artificial Sequence
  Guides targeting the ss Regions in loops in the XIST transcript
tgaggtgggaagatcccttactgccagg

SEQ ID NO: 32 - DNA, 28 bp, Artificial Sequence
  Guides targeting the ss Regions in loops in the XIST transcript
ctcaaactcctggcagtaagggatcttc

SEQ ID NO: 33 - DNA, 28 bp, Artificial Sequence
  Guides targeting the ss Regions in loops in the XIST transcript
gaagatcccttactgccaggagtttgag

SEQ ID NO: 34 - DNA, 28 bp, Artificial Sequence
  Guides targeting the ss Regions in loops in the XIST transcript
taatgttggccaggctggtctcaaactc

SEQ ID NO: 35 - DNA, 28 bp, Artificial Sequence
  Guides targeting the ss Regions in loops in the XIST transcript
gagtttgagaccagcctggccaacatta

SEQ ID NO: 36 - DNA, 28 bp, Artificial Sequence
  Guides targeting the ss Regions in loops in the XIST transcript
taaaggattataaaatttaggtagtttt

SEQ ID NO: 37 - DNA, 28 bp, Artificial Sequence
  Guides targeting the ss Regions in loops in the XIST transcript
aaaactacctaaattttataatccttta

SEQ ID NO: 38 - DNA, 28 bp, Artificial Sequence
  Guides targeting the ss Regions in loops in the XIST transcript
ccagatgaagaaattaaaggattataaa

SEQ ID NO: 39 - DNA, 28 bp, Artificial Sequence
  Guides targeting the ss Regions in loops in the XIST transcript
tttataatcctttaatttcttcatctgg

SEQ ID NO: 40 - DNA, 28 bp, Artificial Sequence
  Guides targeting the ss Regions in loops in the XIST transcript
caggtgctccagatgaagaaattaaagg

SEQ ID NO: 41 - DNA, 28 bp, Artificial Sequence
  Guides targeting the ss Regions in loops in the XIST transcript
cctttaatttcttcatctggagcacctg

SEQ ID NO: 42 - DNA, 28 bp, Artificial Sequence
  Guides targeting the ss Regions in loops in the XIST transcript
aggggcaggtgctccagatgaagaaatt

SEQ ID NO: 43 - DNA, 28 bp, Artificial Sequence
  Guides targeting the ss Regions in loops in the XIST transcript
aatttcttcatctggagcacctgcccct

SEQ ID NO: 44 - DNA, 28 bp, Artificial Sequence
  Guides targeting the ss Regions in loops in the XIST transcript
gaaataagtaggggcaggtgctccagat

SEQ ID NO: 45 - DNA, 28 bp, Artificial Sequence
  Guides targeting the ss Regions in loops in the XIST transcript
atctggagcacctgcccctacttatttc

SEQ ID NO: 46 - DNA, 29 bp, Artificial Sequence
  Guides targeting the ss Regions in loops in the XIST transcript
ttactgcaatcttcttgaaataagtaggg

SEQ ID NO: 47 - DNA, 29 bp, Artificial Sequence
  Guides targeting the ss Regions in loops in the XIST transcript
ccctacttatttcaagaagattgcagtaa

SEQ ID NO: 48 - DNA, 29 bp, Artificial Sequence
  Guides targeting the ss Regions in loops in the XIST transcript
tttactgcaatcttcttgaaataagtagg

SEQ ID NO: 49 - DNA, 29 bp, Artificial Sequence
  Guides targeting the ss Regions in loops in the XIST transcript
cctacttatttcaagaagattgcagtaaa

SEQ ID NO: 50 - DNA, 28 bp, Artificial Sequence
  Guides targeting the ss Regions in loops in the XIST transcript
atgttccctcatttaatcgttttactgc

SEQ ID NO: 51 - DNA, 28 bp, Artificial Sequence
  Guides targeting the ss Regions in loops in the XIST transcript
gcagtaaaacgattaaatgagggaacat

SEQ ID NO: 52 - DNA, 28 bp, Artificial Sequence
  Guides targeting the ss Regions in loops in the XIST transcript
ctgcatatgttccctcatttaatcgttt

SEQ ID NO: 53 - DNA, 28 bp, Artificial Sequence
  Guides targeting the ss Regions in loops in the XIST transcript
aaacgattaaatgagggaacatatgcag

SEQ ID NO: 54 - DNA, 28 bp, Artificial Sequence
  Guides targeting the ss Regions in loops in the XIST transcript
tccctcatttaatcgttttactgcaatc

SEQ ID NO: 55 - DNA, 28 bp, Artificial Sequence
  Guides targeting the ss Regions in loops in the XIST transcript
gattgcagtaaaacgattaaatgaggga

SEQ ID NO: 56 - DNA, 28 bp, Artificial Sequence
  Guides targeting the ss Regions in loops in the XIST transcript
aaggagacatgactactaaggacacatg

SEQ ID NO: 57 - DNA, 28 bp, Artificial Sequence
  Guides targeting the ss Regions in loops in the XIST transcript
catgtgtccttagtagtcatgtctcctt

SEQ ID NO: 58 - DNA, 28 bp, Artificial Sequence
  Guides targeting the ss Regions in loops in the XIST transcript
gactactaaggacacatgcagcgtggta

SEQ ID NO: 59 - DNA, 28 bp, Artificial Sequence
  Guides targeting the ss Regions in loops in the XIST transcript
taccacgctgcatgtgtccttagtagtc

SEQ ID NO: 60 - DNA, 28 bp, Artificial Sequence
  Guides targeting the ss Regions in loops in the XIST transcript
actaaggacacatgcagcgtggtatctt

SEQ ID NO: 61 - DNA, 28 bp, Artificial Sequence
  Guides targeting the ss Regions in loops in the XIST transcript
aagataccacgctgcatgtgtccttagt

SEQ ID NO: 62 - DNA, 28 bp, Artificial Sequence
  Guides targeting the ss Regions in loops in the XIST transcript
ccaattggctcaaaaactaagaatgatt

SEQ ID NO: 63 - DNA, 28 bp, Artificial Sequence
  Guides targeting the ss Regions in loops in the XIST transcript
aatcattcttagtttttgagccaattgg

SEQ ID NO: 64 - DNA, 28 bp, Artificial Sequence
  Guides targeting the ss Regions in loops in the XIST transcript
caaaaactaagaatgattttgaccttat

SEQ ID NO: 65 - DNA, 28 bp, Artificial Sequence
  Guides targeting the ss Regions in loops in the XIST transcript
ataaggtcaaaatcattcttagtttttg

SEQ ID NO: 66 - DNA, 28 bp, Artificial Sequence
  Guides targeting the ss Regions in loops in the XIST transcript
aagaatgattttgaccttataaaaacgt

SEQ ID NO: 67 - DNA, 28 bp, Artificial Sequence
  Guides targeting the ss Regions in loops in the XIST transcript
acgtttttataaggtcaaaatcattctt

SEQ ID NO: 68 - DNA, 28 bp, Artificial Sequence
  Guides targeting the ss Regions in loops in the XIST transcript
ttgaccttataaaaacgttgtttaaaaa

SEQ ID NO: 69 - DNA, 28 bp, Artificial Sequence
  Guides targeting the ss Regions in loops in the XIST transcript
tttttaaacaacgtttttataaggtcaa

SEQ ID NO: 70 - DNA, 28 bp, Artificial Sequence
  Guides targeting the ss Regions in loops in the XIST transcript
gtttaaaaaacaaatatgtaacagaaac

SEQ ID NO: 71 - DNA, 28 bp, Artificial Sequence
  Guides targeting the ss Regions in loops in the XIST transcript
gtttctgttacatatttgttttttaaac

SEQ ID NO: 72 - DNA, 28 bp, Artificial Sequence
  Guides targeting the ss Regions in loops in the XIST transcript
atgtaacagaaaccatatggcccacagt

SEQ ID NO: 73 - DNA, 28 bp, Artificial Sequence
  Guides targeting the ss Regions in loops in the XIST transcript
actgtgggccatatggtttctgttacat

SEQ ID NO: 74 - DNA, 28 bp, Artificial Sequence
  Guides targeting the ss Regions in loops in the XIST transcript
ctaaagtatttatgatttgaccccttac

SEQ ID NO: 75 - DNA, 28 bp, Artificial Sequence
  Guides targeting the ss Regions in loops in the XIST transcript
gtaaggggtcaaatcataaatactttag

SEQ ID NO: 76 - DNA, 28 bp, Artificial Sequence
  Guides targeting the ss Regions in loops in the XIST transcript
ttgaccccttacagaaaaactgtggacc

SEQ ID NO: 77 - DNA, 28 bp, Artificial Sequence
  Guides targeting the ss Regions in loops in the XIST transcript
ggtccacagtttttctgtaaggggtcaa

SEQ ID NO: 78 - DNA, 28 bp, Artificial Sequence
  Guides targeting the ss Regions in loops in the XIST transcript
gaacagcaggccaaatccaattggctca

SEQ ID NO: 79 - DNA, 28 bp, Artificial Sequence
  Guides targeting the ss Regions in loops in the XIST transcript
tgagccaattggatttggcctgctgttc

SEQ ID NO: 80 - DNA, 28 bp, Artificial Sequence
  Guides targeting the ss Regions in loops in the XIST transcript
ggtaagctatgaacagcaggccaaatcc

SEQ ID NO: 81 - DNA, 28 bp, Artificial Sequence
  Guides targeting the ss Regions in loops in the XIST transcript
ggatttggcctgctgttcatagcttacc

SEQ ID NO: 82 - DNA, 28 bp, Artificial Sequence
  Guides targeting the ss Regions in loops in the XIST transcript
acctattggcacccgaatatatttgtag

SEQ ID NO: 83 - DNA, 28 bp, Artificial Sequence
  Guides targeting the ss Regions in loops in the XIST transcript
ctacaaatatattcgggtgccaataggt

SEQ ID NO: 84 - DNA, 28 bp, Artificial Sequence
  Guides targeting the ss Regions in loops in the XIST transcript
ggcacccgaatatatttgtagaatgaat

SEQ ID NO: 85 - DNA, 28 bp, Artificial Sequence
  Guides targeting the ss Regions in loops in the XIST transcript
attcattctacaaatatattcgggtgcc

SEQ ID NO: 86 - DNA, 28 bp, Artificial Sequence
  Guides targeting the ss Regions in loops in the XIST transcript
tataccaagtacctattggcacccgaat

SEQ ID NO: 87 - DNA, 28 bp, Artificial Sequence
  Guides targeting the ss Regions in loops in the XIST transcript
attcgggtgccaataggtacttggtata

SEQ ID NO: 88 - DNA, 28 bp, Artificial Sequence
  Guides targeting the ss Regions in loops in the XIST transcript
ggggccaaaaaccttataccaagtacct

SEQ ID NO: 89 - DNA, 28 bp, Artificial Sequence
  Guides targeting the ss Regions in loops in the XIST transcript
aggtacttggtataaggtttttggcccc

SEQ ID NO: 90 - DNA, 28 bp, Artificial Sequence
  Guides targeting the ds Regions in stem in the XIST transcript
agcggtaggtacactcacacaccaccaa

SEQ ID NO: 91 - DNA, 28 bp, Artificial Sequence
  Guides targeting the ds Regions in stem in the XIST transcript
ttggtggtgtgtgagtgtacctaccgct

SEQ ID NO: 92 - DNA, 28 bp, Artificial Sequence
  Guides targeting the ds Regions in stem in the XIST transcript
atccgccattttggacaacctaacaaag

SEQ ID NO: 93 - DNA, 28 bp, Artificial Sequence
  Guides targeting the ds Regions in stem in the XIST transcript
ctttgttaggttgtccaaaatggcggat

SEQ ID NO: 94 - DNA, 27 bp, Artificial Sequence
  Guides targeting the ds Regions in stem in the XIST transcript
tgaattctacaaataaagcctcttaat

SEQ ID NO: 95 - DNA, 28 bp, Artificial Sequence
  Guides targeting the ds Regions in stem in the XIST transcript
tattaagaggctttatttgtagaattca

SEQ ID NO: 96 - DNA, 28 bp, Artificial Sequence
  Guides targeting the ds Regions in stem in the XIST transcript
gtggccaacacagtacacaagaaaagat

SEQ ID NO: 97 - DNA, 28 bp, Artificial Sequence
  Guides targeting the ds Regions in stem in the XIST transcript
atcttttcttgtgtactgtgttggccac

SEQ ID NO: 98 - DNA, 28 bp, Artificial Sequence
  Guides targeting the ds Regions in stem in the XIST transcript
acaaatacaatcacacatattgggaggc

SEQ ID NO: 99 - DNA, 28 bp, Artificial Sequence
  Guides targeting the ds Regions in stem in the XIST transcript
gcctcccaatatgtgtgattgtatttgt

SEQ ID NO: 100 - DNA, 28 bp, Artificial Sequence
  Guides targeting the ds Regions in stem in the XIST transcript
ccagacgattataatcacacacaacatg

SEQ ID NO: 101 - DNA, 28 bp, Artificial Sequence
  Guides targeting the ds Regions in stem in the XIST transcript
catgttgtgtgtgattataatcgtctgg

SEQ ID NO: 102 - DNA, 28 bp, Artificial Sequence
  Guides targeting the ds Regions in stem in the XIST transcript
actgatgggctgaaaaatgggacaatca

SEQ ID NO: 103 - DNA, 28 bp, Artificial Sequence
  Guides targeting the ds Regions in stem in the XIST transcript
tgattgtcccatttttcagcccatcagt

SEQ ID NO: 104 - DNA, 28 bp, Artificial Sequence
  Guides targeting the ds Regions in stem in the XIST transcript
ttctatccacagacccaccaccctcagt

SEQ ID NO: 105 - DNA, 28 bp, Artificial Sequence
  Guides targeting the ds Regions in stem in the XIST transcript
actgagggtggtgggtctgtggatagaa

SEQ ID NO: 106 - DNA, 28 bp, Artificial Sequence
  Guides targeting the ds Regions in stem in the XIST transcript
cactagaaatcccaaaccccagaatcct

SEQ ID NO: 107 - DNA, 28 bp, Artificial Sequence
  Guides targeting the ds Regions in stem in the XIST transcript
aggattctggggtttgggatttctagtg

SEQ ID NO: 108 - DNA, 28 bp, Artificial Sequence
  Guides targeting the ds Regions in stem in the XIST transcript
caaaattaccagagcagcaaacacaaat

SEQ ID NO: 109 - DNA, 28 bp, Artificial Sequence
  Guides targeting the ds Regions in stem in the XIST transcript
atttgtgtttgctgctctggtaattttg

SEQ ID NO: 110 - DNA, 28 bp, Artificial Sequence
  Guides targeting the ds Regions in stem in the XIST transcript
cggaaaaggtcaaagcccagccaggcct

SEQ ID NO: 111 - DNA, 28 bp, Artificial Sequence
  Guides targeting the ds Regions in stem in the XIST transcript
aggcctggctgggctttgaccttttccg

SEQ ID NO: 112 - DNA, 28 bp, Artificial Sequence
  Guides targeting the ds Regions in stem in the XIST transcript
tgacatttatctatttccttctccactt

SEQ ID NO: 113 - DNA, 28 bp, Artificial Sequence
  Guides targeting the ds Regions in stem in the XIST transcript
aagtggagaaggaaatagataaatgtca

SEQ ID NO: 114 - DNA, 28 bp, Artificial Sequence
  Guides targeting the ss and ds regions in stem- loopjunction in the XIST transcript
gagagaagctgggcgggactgaggaact

SEQ ID NO: 115 - DNA, 28 bp, Artificial Sequence
  Guides targeting the ss and ds regions in stem- loopjunction in the XIST transcript
agttcctcagtcccgcccagcttctctc

SEQ ID NO: 116 - DNA, 28 bp, Artificial Sequence
  Guides targeting the ss and ds regions in stem- loopjunction in the XIST transcript
tttcgagagaagctgggcgggactgagg

SEQ ID NO: 117 - DNA, 28 bp, Artificial Sequence
  Guides targeting the ss and ds regions in stem- loopjunction in the XIST transcript
cctcagtcccgcccagcttctctcgaaa

SEQ ID NO: 118 - DNA, 28 bp, Artificial Sequence
  Guides targeting the ss and ds regions in stem- loopjunction in the XIST transcript
agtgactttcgagagaagctgggcggga

SEQ ID NO: 119 - DNA, 28 bp, Artificial Sequence
  Guides targeting the ss and ds regions in stem- loopjunction in the XIST transcript
tcccgcccagcttctctcgaaagtcact

SEQ ID NO: 120 - DNA, 28 bp, Artificial Sequence
  Guides targeting the ss and ds regions in stem- loopjunction in the XIST transcript
cagggcaattgtcttacttttttttttt

SEQ ID NO: 121 - DNA, 28 bp, Artificial Sequence
  Guides targeting the ss and ds regions in stem- loopjunction in the XIST transcript
aaaaaaaaaaagtaagacaattgccctg

SEQ ID NO: 122 - DNA, 28 bp, Artificial Sequence
  Guides targeting the ss and ds regions in stem- loopjunction in the XIST transcript
aattgtcttacttttttttttttttaat

SEQ ID NO: 123 - DNA, 28 bp, Artificial Sequence
  Guides targeting the ss and ds regions in stem- loopjunction in the XIST transcript
attaaaaaaaaaaaaaagtaagacaatt

SEQ ID NO: 124 - DNA, 28 bp, Artificial Sequence
  Guides targeting the ss and ds regions in stem- loopjunction in the XIST transcript
ttacttttttttttttttaatgttggcc

SEQ ID NO: 125 - DNA, 28 bp, Artificial Sequence
  Guides targeting the ss and ds regions in stem- loopjunction in the XIST transcript
ggccaacattaaaaaaaaaaaaaagtaa

SEQ ID NO: 126 - DNA, 28 bp, Artificial Sequence
  Guides targeting the ss and ds regions in stem- loopjunction in the XIST transcript
atatgctttttaaaagcacctctgcata

SEQ ID NO: 127 - DNA, 28 bp, Artificial Sequence
  Guides targeting the ss and ds regions in stem- loopjunction in the XIST transcript
tatgcagaggtgcttttaaaaagcatat

SEQ ID NO: 128 - DNA, 28 bp, Artificial Sequence
  Guides targeting the ss and ds regions in stem- loopjunction in the XIST transcript
aaaggtggcatatgctttttaaaagcac

SEQ ID NO: 129 - DNA, 28 bp, Artificial Sequence
  Guides targeting the ss and ds regions in stem- loopjunction in the XIST transcript
gtgcttttaaaaagcatatgccaccttt

SEQ ID NO: 130 - DNA, 28 bp, Artificial Sequence
  Guides targeting the ss and ds regions in stem- loopjunction in the XIST transcript
gtggcatatgctttttaaaagcacctct

SEQ ID NO: 131 - DNA, 28 bp, Artificial Sequence
  Guides targeting the ss and ds regions in stem- loopjunction in the XIST transcript
agaggtgcttttaaaaagcatatgccac

SEQ ID NO: 132 - DNA, 28 bp, Artificial Sequence
  Guides targeting the ss and ds regions in stem- loopjunction in the XIST transcript
ggcatatgctttttaaaagcacctctgc

SEQ ID NO: 133 - DNA, 28 bp, Artificial Sequence
  Guides targeting the ss and ds regions in stem- loopjunction in the XIST transcript
gcagaggtgcttttaaaaagcatatgcc

SEQ ID NO: 134 - DNA, 28 bp, Artificial Sequence
  Guides targeting the ss and ds regions in stem- loopjunction in the XIST transcript
ctcacatgctcagaatgtccaagaggag

SEQ ID NO: 135 - DNA, 28 bp, Artificial Sequence
  Guides targeting the ss and ds regions in stem- loopjunction in the XIST transcript
ctcctcttggacattctgagcatgtgag

SEQ ID NO: 136 - DNA, 28 bp, Artificial Sequence
  Guides targeting the ss and ds regions in stem- loopjunction in the XIST transcript
gctcagaatgtccaagaggagcctaagg

SEQ ID NO: 137 - DNA, 28 bp, Artificial Sequence
  Guides targeting the ss and ds regions in stem- loopjunction in the XIST transcript
ccttaggctcctcttggacattctgagc

SEQ ID NO: 138 - DNA, 28 bp, Artificial Sequence
  Guides targeting the ss and ds regions in stem- loopjunction in the XIST transcript
cagaatgtccaagaggagcctaaggaga

SEQ ID NO: 139 - DNA, 28 bp, Artificial Sequence
  Guides targeting the ss and ds regions in stem- loopjunction in the XIST transcript
tctccttaggctcctcttggacattctg

SEQ ID NO: 140 - DNA, 28 bp, Artificial Sequence
  Guides targeting the ss and ds regions in stem- loopjunction in the XIST transcript
ggtctcacatgctcagaatgtccaagag

SEQ ID NO: 141 - DNA, 28 bp, Artificial Sequence
  Guides targeting the ss and ds regions in stem- loopjunction in the XIST transcript
ctcttggacattctgagcatgtgagacc

SEQ ID NO: 142 - DNA, 28 bp, Artificial Sequence
  Guides targeting the ss and ds regions in stem- loopjunction in the XIST transcript
gaataacaaataatacatcagggggtgg

SEQ ID NO: 143 - DNA, 28 bp, Artificial Sequence
  Guides targeting the ss and ds regions in stem- loopjunction in the XIST transcript
ccaccccctgatgtattatttgttattc

SEQ ID NO: 144 - DNA, 28 bp, Artificial Sequence
  Guides targeting the ss and ds regions in stem- loopjunction in the XIST transcript
aatacatcagggggtggtaagctatgaa

SEQ ID NO: 145 - DNA, 28 bp, Artificial Sequence
  Guides targeting the ss and ds regions in stem- loopjunction in the XIST transcript
ttcatagcttaccaccccctgatgtatt

SEQ ID NO: 146 - DNA, 28 bp, Artificial Sequence
  Guides targeting the ss and ds regions in stem- loopjunction in the XIST transcript
aaataatacatcagggggtggtaagcta

SEQ ID NO: 147 - DNA, 28 bp, Artificial Sequence
  Guides targeting the ss and ds regions in stem- loopjunction in the XIST transcript
tagcttaccaccccctgatgtattattt

SEQ ID NO: 148 - DNA, 28 bp, Artificial Sequence
  Guides targeting the ss and ds regions in stem- loopjunction in the XIST transcript
aataacaaataatacatcagggggtggt

SEQ ID NO: 149 - DNA, 27 bp, Artificial Sequence
  Guides targeting the ss and ds regions in stem- loopjunction in the XIST transcript
accaccccctgatgtattatttgttat

SEQ ID NO: 150 - DNA, 28 bp, Artificial Sequence
  Guides targeting the ss and ds regions in stem- loopjunction in the XIST transcript
ggaaggcatgcatttttttcccatgtct

SEQ ID NO: 151 - DNA, 28 bp, Artificial Sequence
  Guides targeting the ss and ds regions in stem- loopjunction in the XIST transcript
agacatgggaaaaaaatgcatgccttcc

SEQ ID NO: 152 - DNA, 28 bp, Artificial Sequence
  Guides targeting the ss and ds regions in stem- loopjunction in the XIST transcript
ctctgggaaggcatgcatttttttccca

SEQ ID NO: 153 - DNA, 28 bp, Artificial Sequence
  Guides targeting the ss and ds regions in stem- loopjunction in the XIST transcript
tgggaaaaaaatgcatgccttcccagag

SEQ ID NO: 154 - DNA, 28 bp, Artificial Sequence
  Guides targeting the ss and ds regions in stem- loopjunction in the XIST transcript
gcatgcatttttttcccatgtctctggg

SEQ ID NO: 155 - DNA, 28 bp, Artificial Sequence
  Guides targeting the ss and ds regions in stem- loopjunction in the XIST transcript
cccagagacatgggaaaaaaatgcatgc

SEQ ID NO: 156 - DNA, 28 bp, Artificial Sequence
  Guides targeting the ss and ds regions in stem- loopjunction in the XIST transcript
catgcatttttttcccatgtctctgggg

SEQ ID NO: 157 - DNA, 28 bp, Artificial Sequence
  Guides targeting the ss and ds regions in stem- loopjunction in the XIST transcript
ccccagagacatgggaaaaaaatgcatg

(19) United States
(12) Patent Application Publication
(10) Pub. No.: US 2021/0189386 A1
(43) Pub. Date: Jun. 24, 2021

(54) NUCLEIC ACID CONSTRUCT, MEDICINAL COMPOSITION, ANTICANCER AGENT, ANTIVIRAL AGENT AND ANTIBACTERIAL AGENT

(71) Applicant: POiRT Systems Co., Ltd., Okayama (JP)

(72) Inventors: Tatsuo ITO, Okayama (JP); Keiki OGINO, Kochi (JP); Mika HIGASHIDE, Fukui (JP); Srinivas BANDARU, Okayama (JP); Yurika SHIMIZU, Okayama (JP)

(73) Assignee: POiRT Systems Co., Ltd., Okayama (JP)

(21) Appl. No.: 17/043,138

(22) PCT Filed: Mar. 29, 2019
(86) PCT No.: PCT/JP2019/014183

§ 371 Date: Sep. 29, 2020

(30) Foreign Application Priority Data
Mar. 30, 2018 (JP) 2018-067565

(51) Int. Cl. C12N 15/11; C12N 9/22; C12N 15/85

(52) U.S. Cl. CPC C12N 15/11; C12N 9/22; C12N 2800/80; C12N 2310/20; C12N 15/85

(57) ABSTRACT

The present invention provides a nucleic acid construct containing at least one guide RNA portion that binds to one or more target RNAs and an RNA-cleaving Cas protein expression portion, wherein the one or more target RNAs are derived from a mutation in a vertebrate cell, a virus, or a bacterium.

### TECHNICAL FIELD

The present invention relates to a nucleic acid construct, a pharmaceutical composition, an anticancer agent, an antiviral agent, and an antibacterial agent.

### BACKGROUND ART

For genome editing, many application examples are known as CRISPR/Cas9 systems. Genome editing is performed by simultaneously delivering CRISPR/Cas9 enzymes or an expression construct encoding CRISPR/Cas9 to target cells, together with guide nucleic acids. The application of genome editing in therapy has drawbacks in terms of serious side effects (Non-patent Literature (NPL) 1).

In addition to genome editing, RNA editing is also known. NPL 2 and NPL 3 disclose C2C2/Cas13 as an RNA editing enzyme.

Regulation of mRNA expression in cells has been attempted in cancer therapy using siRNA and shRNA since the early 2000s (NPL 4 and NPL 5). Although this technology had a significant impact on the regulation of protein gene expression in basic study, no effective use for therapy or diagnosis was found in clinical settings including cancer therapy. This is because both siRNA and shRNA molecules consist only of nucleic acid (RNA), the selectivity for target genes was low, and versatility was insufficient in affecting therapeutic targets.

Antiviral agents and antibacterial agents have been developed for infectious diseases; however, the use of these agents causes a problem in terms of resistance.

### CITATION LIST

### Non-Patent Literature

NPL 1: Kellie A Schaefer, Wen-Hsuan Wu, Diana F Colgan, Stephen H Tsang, Alexander G Bassuk, VinitB Mahajan. “Unexpected mutations after CRISPR-Cas9 editing in vivo.” Nature Methods. 2017, 14, 547-548
NPL 2: Wright A V, Nunez J K, Doudna J A. Cell. 2016 Jan. 14; 164 (1-2): 29-44
NPL 3: Gootenberg J S, Abudayyeh O O, Kellner M J, Joung J, Collins J J, Zhang F. “Multiplexed and portable nucleic acid detection platform with Cas13, Cas12a, and Csm6.” Science. 2018 Feb. 15.
NPL 4: Paddison P J, Caudy A A, Bernstein E, Hannon G J, Conklin D S. Genes & Development (2002). 16 (8): 948-58.
NPL 5: Hamilton A, Baulcombe D (1999), “A species of small antisense RNA in posttranscriptional gene silencing in plants.” Science. 286 (5441): 950-2.

### SUMMARY OF INVENTION

### Technical Problem

An object of the present invention is to provide therapeutic techniques for cancer and infectious diseases caused by viruses or bacteria.

### Solution to Problem

The present invention provides the following nucleic acid construct, pharmaceutical composition, anticancer agent, antiviral agent, and antibacterial agent.

### Item 1.

A nucleic acid construct comprising at least one guide RNA portion that binds to one or more target RNAs and an RNA-cleaving Cas protein expression portion, wherein the cne or more target RNAs are derived from a mutation in a vertebrate cell, a virus, or a bacterium.

### Item 2.

The nucleic acid construct according to Item 1, wherein the at least one guide RNA portion and the RNA-cleaving Cas protein expression portion are present in a single nucleic acid sequence.

### Item 3.

The nucleic acid construct according to Item 1, comprising two or more nucleic acids,

wherein the at least one guide RNA portion and the RNA-cleaving Cas protein expression portion are present in separate nucleic acid sequences.

### Item 4.

The nucleic acid construct according to any one of Items 1 to 3, which is an RNA construct or a DNA construct.

### Item 5.

The nucleic acid construct according to any one of Items 1 to 4, wherein an RNA-cleaving Cas protein is a Cas13 family protein.

### Item 6.

The nucleic acid construct according to any one of Items 1 to 5, wherein an RNA-cleaving Cas protein is C2C2/Cas13a.

### Item 7.

The nucleic acid construct according to any one of Items 1 to 6, wherein at least one guide RNA targets RNA that corresponds to a mutation in a vertebrate cell.

### Item 8.

The nucleic acid construct according to any one of Items 1 to 7, wherein

the mutation in a vertebrate cell is a translocation, and

at least one guide RNA targets RNA that corresponds to a gene of the translocation.

### Item 9.

The nucleic acid construct according to any one of Items 1 to 6, wherein the virus is one member selected from the group consisting of an influenza virus, an HIV virus, a herpesvirus, an Ebola virus, an avian influenza virus, a foot-and-mouth disease virus, a SARS coronavirus, a MERS coronavirus, a papillomavirus, a hepatitis virus (hepatitis virus A, B, and C), a measles virus, a rubella virus, a mumps virus, a rotavirus, an RS virus, a norovirus, a herpes zoster virus, a poliovirus, a dengue virus, and an adult T-cell leukemia virus.

### Item 10.

A pharmaceutical composition, comprising the nucleic acid construct of any one of Items 1 to 8 as an active ingredient.

### Item 11.

An anticancer agent, comprising the nucleic acid construct of any one of Items 1 to 8 as an active ingredient.

### Item 12.

An antiviral agent, comprising the nucleic acid construct of any one of Items 1 to 8 as an active ingredient.

### Item 13.

An antibacterial agent, comprising the nucleic acid construct of any one of Items 1 to 8 as an active ingredient.

### Advantageous Effects of Invention

The present invention uses an RNA gene modification technique and is superior to known art in terms of the degree of freedom in selecting a target gene and specificity to the target gene.

The nucleic acid construct according to the present invention indiscriminately reduces the expression of mRNA in cancer cells, virally infected cells, or in bacteria, and does not substantially act on normal cells. Thus, the nucleic acid construct reduces side effects and exhibits a more potent antitumor effect, antiviral effect, or antimicrobial effect than known art. Additionally, the nucleic acid construct can be used in combination with conventional therapeutic methods such as anticancer agents, antimicrobial drugs, or antiviral drugs.

The nucleic acid construct according to the present invention is a transitory effect development mechanism and involves no genome invasion, thus exhibiting less invasion to normal cells than known art.

### BRIEF DESCRIPTION OF DRAWINGS

FIG. 1: Changes in the survival rate due to RNA cleavage specific to synovial sarcoma-specific synovial sarcoma X chromosome (SSX) fusion gene. For SSX, 5 types of guide RNAs were created using the reported fusion site of synovial sarcoma SYO-1 cells as the target, including a negative control (NC). The bold letters (C, G, C, C, and A, each respectively referred to as “SSX1” to “SSX5”) represent PAM sequences. SYO-1 represents cells at 40% confluency. Guide 1 (SSX-1, terminal: C), guide 2 (SSX-2, negative control (nc), terminal: G), guide 3 (SSX-3, terminal: C), guide 4 (SSX-4, terminal: C), and guide 5 (SSX-5, terminal: A).

FIG. 2: Target gene for therapy 1: synovial sarcoma-specific translocation gene t(X;18) (p11.2;q11.2) as a target for therapy. The increase in the dead cell percentage was notable in Guide_SSX-3, SSX-4, and SSX-5.

FIG. 3: Specific RNA cleavage by C2c2_Lsh: Experiment for confirming cleavage of translocation gene cDNA of brain tumor.

FIG. 4: The results of northern blotting on the cleavage of brain tumor (epithelioma)-specific translocation gene C11orf95-RELA (11q13.1) cDNA. Lane 1 shows the electrophoresis results of a substance with a standard molecular weight. Lanes 2 to 10 show the results of guide RNAs 1 to 9 of FIG. 3. Lane NC shows the results of guide RNA 10nc of FIG. 3.

FIG. 5: crRNA Design based on RNA structure (ssRNA vs dsRNA).

FIG. 6: Diagram explaining a PA magnet system.

FIG. 7: Diagram explaining a PA magnet system. Quantitation of gDNA binding to XIST.

FIG. 8: Target RNA editing from C to U by a RESCUE (RNA Engineering by Substitution of Cytidine to Uridine Edits) system.

FIG. 9: Reduction in AP production by inhibiting 3-secretase cleavage by the RESCUE system.

### DESCRIPTION OF EMBODIMENTS

The nucleic acid construct according to the present invention may be either DNA or RNA, and may contain both DNA and RNA.

The nucleic acid construct according to the present invention contains (1) at least one guide RNA portion that binds to one or more target RNAs and (2) an RNA-cleaving Cas protein expression portion. The guide RNA (gRNA) portion as used here refers to guide RNA itself when the nucleic acid is RNA. The guide RNA portion refers to DNA capable of expressing guide RNA in a cell into which the nucleic acid construct is introduced when the nucleic acid is DNA. The “guide RNA portion” includes both guide RNA itself and DNA capable of expressing guide RNA in meaning, and may be either one or both. The RNA-cleaving Cas protein expression portion refers to RNA capable of expressing an RNA-cleaving Cas protein (e.g., a portion that corresponds to mRNA containing the post-splicing coding region of an RNA-cleaving Cas protein) when the nucleic acid is RNA. The RNA-cleaving Cas protein expression portion refers to DNA capable of expressing an RNA-cleaving Cas protein (e.g., DNA that contains a promoter and a coding region of the RNA-cleaving Cas protein (introns may be contained)) when the nucleic acid is DNA. The RNA-cleaving Cas protein expression portion may be composed of one portion of DNA or RNA encoding an RNA-cleaving Cas protein;

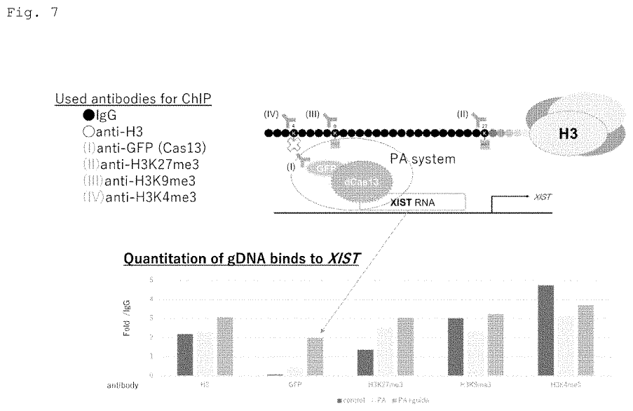

the DNA or RNA encoding an RNA-cleaving Cas protein may be divided into two or more portions such that their expression products collaborate intracellularly to exhibit RNA-cleaving activity (e.g., the system illustrated in FIG. 7).

In the present specification, “a mutation in a vertebrate cell” refers to, for example, translocation, inversion, or deletion or insertion of multiple bases, and is a mutation associated with cancerization; RNA derived from a mutation is produced in cancer cells of a vertebrate, and not produced in normal cells. This mutation is present in post-splicing RNA, and does not include a mutation of an intron. The mutation in the present invention does not also include single-nucleotide polymorphisms (SNPs). In an embodiment of the present invention, RNA derived from a mutation is a target with which a guide RNA hybridizes. In another embodiment of the present invention, a target RNA is produced in a vertebrate cell infected with a target virus, and not produced in a cell uninfected with the target virus. Further, in another embodiment of the present invention, a target RNA is produced in a target bacterium, and not produced in a vertebrate cell including a human cell.

The guide RNA contains a sequence complementary to a target RNA and a PAM sequence. The guide RNA for use can be those used in genome editing. The number of bases of the sequence complementary to a target RNA is 20 to 30, preferably 22 to 30, more preferably 24 to 29, still more preferably 26 to 29, and most preferably 28. The PAM sequence depends on the origin and type of the organism from which the RNA-cleaving Cas protein is derived. The PAM sequence is, for example, more preferably A, but may be C or U. In the present invention using RNA editing, the PAM sequence is different from that of genome editing using Cas9, and the PAM sequence for use in RNA editing is short.

When the nucleic acid construct is RNA, the loop portion of the guide RNA may be formed beforehand. The guide RNA may be sgRNA in which crRNA (CRISPR RNA) and tract RNA (trans-activating RNA) are linked to each other, or may be guide RNA prepared by synthesizing crRNA and tract RNA as separate RNAs and hybridizing these RNAs to form a complex.

Examples of RNA-cleaving Cas proteins include Cas13 family proteins. The RNA-cleaving Cas protein is preferably, for example, Cas13a/C2C2, Cas13b, or Cas13c, and more preferably Cas13a/C2C2. “Cas13a” and “C2C2” both refer to the same RNA-cleaving Cas protein.

When the nucleic acid construct according to the present invention is DNA, the nucleic acid construct can be incorporated into a plasmid or a virus vector. When a nucleic acid construct in the form of DNA is used, intracellular transcription occurs to form a nucleic acid construct in the form of RNA, thereby forming an RNA-cleaving Cas protein and guide RNA in cytoplasm. When a single nucleic acid construct contains a nucleic acid (DNA or RNA) capable of expressing an RNA-cleaving Cas protein and guide RNA, the RNA-cleaving Cas protein and the guide RNA are preferably linked via a hammerhead ribozyme (HHR) sequence. When the nucleic acid construct according to the present invention contains multiple guide RNAs, adjacent guide RNAs are preferably linked via a hammerhead ribozyme sequence. The hammerhead ribozyme sequence is intracellularly cleaved by the self-cleavage function, and each guide RNA as well as RNA capable of expressing the RNA-cleaving Cas protein are produced in cells.

The hammerhead ribozyme (HHR) can cleave the RNA phosphodiester bond in a specific site, and a minimal hammerhead ribozyme that cleaves a trans-cleaving ribozyme is prepared by modifying natural HHR. The hammerhead ribozyme is used in reducing the expression of a target gene in vivo by RNA-mediated gene regulation.

The expression product of the nucleic acid construct according to the present invention contains one or more guide RNAs and an RNA-cleaving Cas protein. These guide RNAs and RNA-cleaving Cas protein act in cytoplasm of target vertebrate cells (cancer cells or virally infected cells) and/or of a bacterium. Specifically, when a target RNA that hybridizes with a guide RNA is present in cytoplasm, the guide RNA forms a hybrid with the target RNA, which leads the RNA-cleaving Cas protein to cleave and decompose not only the hybrid RNA but also RNAs present around in the cytoplasm, thereby killing the cells. For example, cells that have become cancerous by chromosome translocation contain RNA that corresponds to the translocation in the cells. Thus, introducing the nucleic acid construct of the present invention into the cancer cells kills the cancer cells, but does not affect normal cells because normal cells involve no translocation. Thus, introducing the nucleic acid construct of the present invention into the cells of a whole body of a vertebrate kills only cancer cells, while the nucleic acid construct decomposes in the cytoplasm, thus causing almost no side effects or toxicity to the normal cells. The case above has been explained with translocation as an example of mutations. However, the nucleic acid construct of the present invention can also selectively kill cancer cells caused by other mutations such as inversion, insertion, or deletion, as long as no target RNA is present in normal cells, and the target RNA is present only in cancer cells. RNAs to which Cas13 binds as a target are preferably composed of a short-chain sequence. Thus, in extracting and designing a crRNA sequence, optimal sequence extraction based on the prediction of a target RNA sequence and conformation is required in the present invention.

In a preferable embodiment according to the present invention, the RNA-cleaving Cas protein can be divided into two components. The functional activity of the RNA-cleaving Cas protein can be regulated externally by fusing each divided fragment with a PA magnet system for fusing protein fragments that form a dimer depending on specific wavelength stimulation (research paper for reference: Yuta Nihongaki et al., “Photoactivatable CRISPR-Cas9 for optogenetic genome editing,” Nature Biotechnology, Published online 15 Jun. 2015). The use of this mechanism enables the regulation of RNA-cleaving Cas protein functions in a site affected after the uptake of the divided fragments in vivo by using an adeno-associated virus (AAV) vector (the site irradiated with light from an optical source embedded in vivo). For example, β-secretase is known to cleave amyloid precursor protein (APP) to produce amyloid β-protein (Aβ). With the system in which mRNA of β-secretase is the target RNA, and the production of β-secretase is inhibited in the hippocampus only during irradiation with light from the light source embedded in the hippocampus, Alzheimer's disease can be treated, reducing side effects. This is because the inhibition of β-secretase can be regulated by light irradiation.

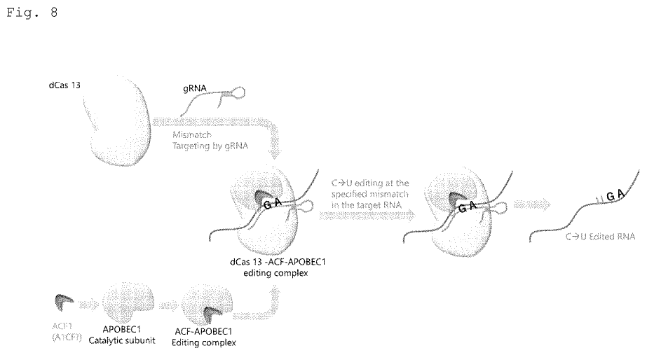

In a preferable embodiment according to the present invention, an RNA-cleaving Cas protein, which is the expression product of the nucleic acid construct of the present invention, can edit RNA by using an RNA-cleaving active mutant (FIG. 8). Specifically, RNA-non-cleaving Cas13 (dCas13) is fused with the active site of an RNA-editing enzyme (APOBEC1), and the RNA-condensation-activating domain of A1CF protein, which is a coenzyme of APOBEC protein, is further addition-fused thereto. The target RNA hauled in by Cas13 and crRNA is presented by the A1CF domain to the APOBEC1 domain, and the RNA sequence in a specific region is edited (editing C→U in FIG. 8).

In the present specification, vertebrates include humans, chimpanzees, monkeys, cows, horses, swine, sheep, rabbits, mice, rats, dogs, cats, chickens, wild ducks, and domesticated ducks, with humans, domestic animals (e.g., cows, swine, and chickens), and pets (e.g., dogs and cats) being preferable.

When the guide RNA according to the present invention forms a hybrid with RNA derived from a virus, but not with RNA of vertebrate cells, only vertebrate cells infected with the virus are killed, and non-virally infected cells are not affected. Thus, the nucleic acid construct according to the present invention can treat virus infection without causing serious side effects.

When the guide RNA according to the present invention forms a hybrid with RNA derived from a bacterium, but not with RNA of vertebrate cells, only the bacterium is killed, thereby causing almost no toxicity to the vertebrate. Thus, the nucleic acid construct according to the present invention can treat bacterial infection. Additionally, the nucleic acid construct according to the present invention is useful as an antimicrobial cleaning agent or disinfectant. The antimicrobial action of the nucleic acid construct according to the present invention is also useful for antibiotic drug-resistant bacteria such as multidrug-resistant bacteria because such bacteria do not develop resistance to the antimicrobial action of the nucleic acid construct.

The nucleic acid construct according to the present invention may contain in a single nucleic acid sequence (1) at least one guide RNA that binds to one or more target RNAs or DNA encoding the at least one guide RNA and (2) RNA encoding an RNA-cleaving Cas protein or DNA encoding the RNA (the RNA or DNA may be composed of a single sequence or of divided two or more sequences). The nucleic acid construct according to the present invention may also contain in separate nucleic acid sequences (1) the guide RNA or DNA encoding the guide RNA and (2) RNA encoding an RNA-cleaving Cas protein or DNA encoding the RNA. In this case, the nucleic acid construct according to the present invention is a composition containing multiple nucleic acid sequences.

Multiple types of guide RNAs or multiple guide RNAs of an identical type may be present. Additionally, several guide RNAs that target the same RNA but that are composed of different nucleic acid sequences may be present. For example, when there are multiple translocation sequences involved in cancerization, introducing multiple guide RNAs into vertebrate cells kills multiple types of cancer cells at the same time. When multiple target RNAs are produced by a single translocation site, multiple guide RNAs can kill cancer cells more reliably.

By containing multiple types of guide RNAs that correspond to the sequences unique to many different types of influenza viruses (e.g., influenza A virus and B virus), the nucleic acid construct according to the present invention can provide an antiviral agent effective against all types of influenza viruses that became epidemic in the past. Additionally, by containing multiple types of guide RNAs that correspond to the sequences unique to many types of bacteria (in particular, pathogenic bacteria), the nucleic acid construct according to the present invention can provide, for example, an antibacterial agent or disinfectant effective against many bacterial infectious diseases. A viral or bacterial infectious disease can be caused by only one type of virus or bacterium. However, a viral or bacterial infectious disease can also be caused by simultaneous infection with multiple types of viruses and/or bacteria in some cases. The use of guide RNAs that address multiple types of viruses and/or bacteria enables the treatment of viral or bacterial infectious diseases without strictly specifying the kind and type of viruses or bacteria.

Cancers treatable with the nucleic acid construct according to the present invention include cancers caused by gene mutation, such as synovial sarcoma, brain tumor, leukemia, malignant lymphoma, lung cancer, prostate cancer, and renal cell cancer. The nucleic acid construct according to the present invention can kill only cancer cells in which RNA involved in translocation is present. Cancerization involves gene mutation. Cancer cells in which RNA unique to gene mutation is produced can be killed all together with cells that have become cancerous by mutations other than translocation by the nucleic acid construct according to the present invention that contains at least one guide RNA that corresponds to the unique RNA. The anticancer agent according to the present invention can be used in both the primary focus and the metastatic focus, and can also be used in the prevention of recurrence after surgery. The anticancer agent according to the present invention can also be used in combination with at least one other anticancer agent.

The nucleic acid construct according to the present invention is also useful as a therapeutic agent for Alzheimer's disease by regulating the production of amyloid β-protein.

In a preferable embodiment according to the present invention, due to lack of the nuclear localization signal, the nucleic acid construct containing RNA according to the present invention does not move into a nucleus, making no direct action on the chromosomes, DNA, and genes. This is one reason why the nucleic acid construct has a low degree of side effects.

In a preferable embodiment according to the present invention, the nucleic acid construct of the present invention is introduced into vertebrate cells or bacteria, in particular, the cytoplasm. In the case of a vertebrate, the introducing agent for introducing a nucleic acid construct such as RNA or DNA into cells is not particularly limited; any known introducing agent is usable. For example, the introducing agent is a liposome, exosome, liposome-exosome hybrid, Sendai virus, or virus vector (e.g., an adenovirus vector), preferably an exosome, Sendai virus, or virus vector, and particularly preferably an exosome. An exosome is suitable for use in introducing a nucleic acid construct into cells (e.g., cancer cells and virally infected cells) because the nucleic acid construct can be easily introduced into cells by mixing an exome with the nucleic acid construct. The nucleic acid construct according to the present invention includes pharmaceutical compositions that contain the introducing agent described above and the nucleic acid construct. When a bacterium is targeted, the nucleic acid construct can be dissolved, dispersed, or suspended in a calcium-ion-containing medium in order to introduce the nucleic acid construct into the cells of the bacterium. Examples of such a medium include water, buffers, and water-miscible organic solvents, such as ethanol.

Examples of viruses targeted by the antiviral agent include influenza viruses (including influenza A virus and B virus), HIV virus, herpesvirus, Ebola virus, avian influenza virus, foot-and-mouth disease virus, SARS coronavirus, MERS coronavirus, papillomavirus, hepatitis viruses (hepatitis A virus, B virus, and C virus), measles virus, rubella virus, mumps virus, rotavirus, RS virus, norovirus, herpes zoster virus, poliovirus, dengue virus, Zika virus, and adult T-cell leukemia virus.

Examples of bacteria targeted by the antibacterial agent include Shigella, Mycobacterium tuberculosis, cholera bacillus, serratia, vulnificus, aeromonad, pertussis, Brucella, Bartonella, Legionella pneumophila, Coxiella, gonococcal, campylobacter, Helicobacter pylori, Staphylococcus aureus, Streptococcus pyogenes, anthrax, gas gangrene, Clostridium botulinum, Listeria monocytogenes, Corynebacterium diphtheriae, mycoplasma, Chlamydia pneumonia, pneumococcus, Clostridium tetani, Yersinia pestis, enterohemorrhagic Escherichia coli (e.g., 0157), Vibrio parahaemolyticus, Salmonella, Clostridium welchii, hemolytic Streptococcus, meningococcus, Proteobacteria, Pseudomonas aeruginosa, Citrobacter, Acinetobacter, Enterobacter, Klebsiella, Clostridium, and Trichophyton fungi.

The nucleic acid construct according to the present invention used as a medical drug (e.g., anticancer agents, antiviral agents, and antibacterial agents) can be administered at a dose of about 1 ng to 1000 mg per day for an adult, once daily or in 2 to 4 divided doses daily. Examples of dosage forms of the medical drug include injectable drugs, tablets, capsules, inhalants, fluid medicines, drinkable preparations, suppositories, spray agents, plasters, ointments, and ophthalmic solutions.

### EXAMPLES

The present invention will be described in more detail below based on Examples.

### Example 1

### (1) Plasmid Construction and Target Guide

The DNA sequence of C2C2 Lsh (Leptotrichia shahii) was amplified and fused with the pX458 plasmid using Hifi DNA Assembly (NEB). An Lsh-specific scaffold RNA sequence was inserted using Hifi DNA Assembly (NEB). Phosphorylated oligonucleotides encoding an sgRNA sequence were ligated to Bbs1-digested scaffold constructs to prepare sgRNA-targeting XistRNAs, C11orf95-RELA fusion RNAs, and SS18-SSX fusion RNAs. The platform thereof was named “pLMT” (pLMTXist plasmids). The 15 different pLMTXist plasmids each contained any of the 15 types of guide RNAs shown in Table 1.

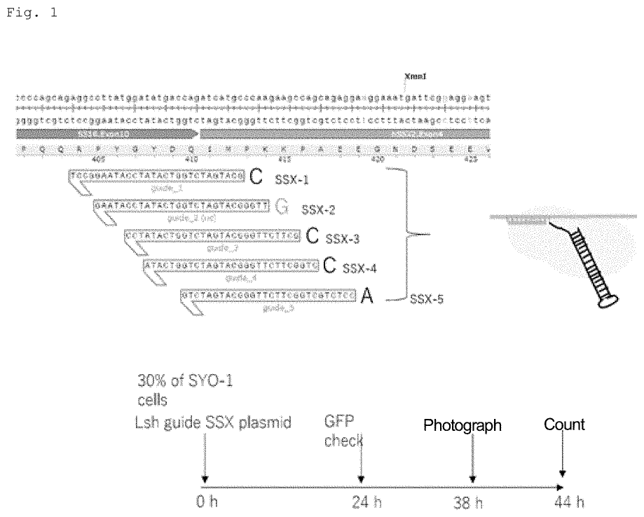

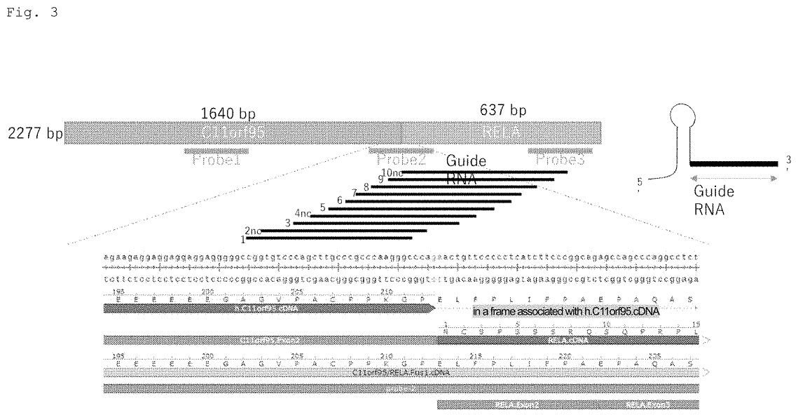

Table 1 below shows the sequences of 5 different guide RNAs introduced into synovial sarcoma cells (SYO-1 containing a SS18-SSX fusion gene) (SSX-1, SSX-2, SSX-3, SSX-4, and SSX-5), and 10 different guide RNAs introduced into epithelioma cells (HEK293T into which a translocation gene C11orf95-RELA (11q13.1) associated with brain tumor was introduced). FIG. 1 shows 5 different guide RNAs introduced into the synovial sarcoma cells, and the experimental conditions. FIG. 3 shows 10 different guide RNAs introduced into the HEK293T. The sequences in Tables 1 to 4 show the genetic information of the DNA templates.

TABLE 1

| | h.C11orf95RELA fusion |
|---|---|
| Epithelioma | Guide RNA list |
| Guide1 | GCCCTTGGGCGGGCAAGCTGGGACACCG |
| Guide2nc | TGGGCCCTTGGGCGGGCAAGCTGGGACA |
| Guide3 | TTCTGGGCCCTTGGGCGGGCAAGCTGGG |
| Guide4nc | AGTTCTGGGCCCTTGGGCGGGCAAGCTG |
| Guide5 | GGGAACAGTTCTGGGCCCTTGGGCGGGC |
| Guide6 | AGGGGGAACAGTTCTGGGCCCTTGGGCG |
| Guide7 | ATGAGGGGGAACAGTTCTGGGCCCTTGG |
| Guide8 | AAGATGAGGGGGAACAGTTCTGGGCCCT |
| Guide9 | GGGAAGATGAGGGGGAACAGTTCTGGGC |
| Guide 10n.C. | AACAGTTCTGGGCCCTTGGGCGGGCAAG |
| Synovial | SS18.SSX fusion Guide |
| sarcoma | Guide RNA List |
| Guide_1 | GCATGATCTGGTCATATCCATAAGGCCT |
| Guide_2 (nC) | TTGGGCATGATCTGGTCATATCCATAAG |
| Guide_3 | GCTTCTTGGGCATGATCTGGTCATATCC |
| Guide_4 | CTGGCTTCTTGGGCATGATCTGGTCATA |
| Guide_5 | CCTCTGCTGGCTTCTTGGGCATGATCTG |

### (2) Cell Culture

The HEK293T and SYO-1 synovial sarcoma cell lines were maintained in D-MEM (low-glucose) medium supplemented with 1% penicillin, streptomycin, and 10% fetal bovine serum (FBS). The obtained cells were cultured in a humidified atmosphere at 37° C. in 5% CO₂.

The HEK293T was obtained by introducing a translocation gene C11orf95-RELA (11q13.1) associated with brain tumor. The SYO-1 contained a SS18-SSX fusion gene.

### (3) Northern Blotting

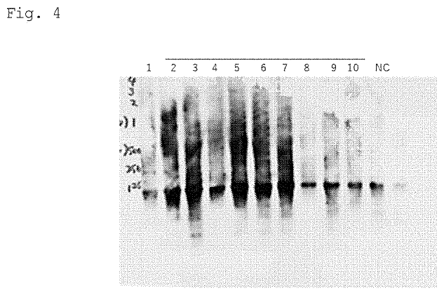

The HEK293T cells at 30% confluency were transfected with the pLMT_Xist plasmids using ScreenFect A (Wako). The 10 different pLMT_Xist plasmids each contained any guide RNA selected from the “Epithelioma h.C11orf95RELA fusion Guide RNA list” in Table 1. After being cultured for 48 hours, the cells were harvested. The RNAs were precipitated using ISOGEN. Northern blotting was performed using a DIG Northern Starter kit (Roche). The membrane was hybridized with a DIG-labeled probe targeting XistRNA and C11-orf95-RELA in a hybridization buffer (7% SDS, 0.5 M Na-phosphate buffer (pH 7.2), 10 mM EDTA), and washed with a washing buffer (1% SDS, Na-phosphate buffer (pH 7.2), 10 mM EDTA). FIG. 4 shows the results of northern blotting.

### (4) Trypan Blue Assay

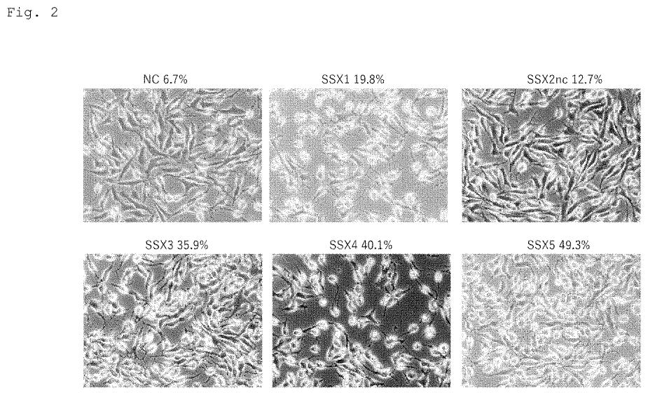

The SYO-1 cells at 30% confluency were transfected with the pLMT_Xist plasmids by using ScreenFect A (Wako). After being cultured for 48 hours, the cells were harvested. The cell suspension and a trypan blue solution, 0.4%, were mixed at 1:1. The live cells and dead cells were counted with a hemocytometer to determine the proportion of dead cells. FIG. 2 shows the results.

### Example 2

### (1) Plasmid Construction and Target Guide

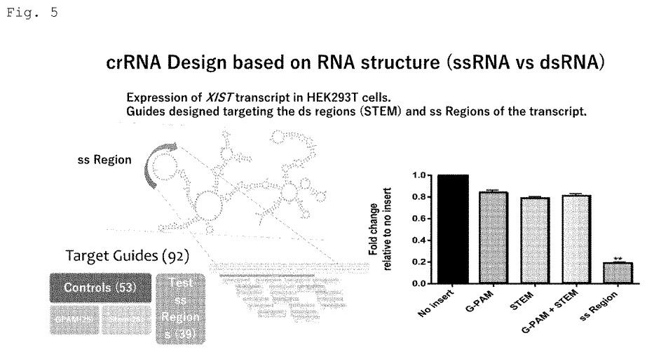

The DNA sequence of C2C2 Lsh (Leptotrichia shahii) was amplified and fused with the pX458 plasmid using Hifi DNA Assembly (NEB). An Lsh-specific scaffold RNA sequence was inserted using Hifi DNA Assembly (NEB). A phosphorylated oligonucleotide encoding an sgRNA sequence was ligated to Bbs1-digested scaffold constructs to prepare sgRNA-targeting XistRNAs and fusion RNAs. Tables 2 to 4 below show 71 different guide RNA sequences designed based on RNA conformation prediction and introduced into the HEK293T. FIG. 5 shows the 71 introduced guide RNAs, the experimental conditions, and the results.

TABLE 2
Guides targeting the ss Regions in loops in the XIST trascript
(Total crRNAs = 37)

| SENSE | ANTISENSE | EXON | TARGET SITE |
|---|---|---|---|
| AGACTAGGGGTTTGCTGGGAGCAGGGCT | AGCCCTGCTCCCACAAACCCCTAGTCT | 1 | 2816-2823 |
| GGGGCTAGACTAGGGGTTTGCTGGGAGC | GCTCCCAGCAAACCCCTAGTCTAGCCCC | 1 | 2822-2830 |
| GGGGGGTTAGGGGACTGGGGCTGGGGCA | TGCCCCAGCCCCAGTCCCCTAACCCCCC | 1 | 2877-2904 |
| GGACTGGGGCTAGGGCTGGGGGGTTAG | CTAACCCCCCAGCCCTAGCCCCAGTCCC | 1 | 2895-2922 |
| GCTGGGATTACAGGTGTGAGCCACCACA | TGTGGTGGCTCACACCTGTAATCCCAGC | 1 | 5242-5269 |
| CCCAAAGTGCTGGGATTACAGGTGTGAG | CTCACACCTGTAATCCCAGCACTTTGGG | 1 | 5250-5277 |
| AAGGGATCTTCCCACCTCAGCCTCCCAA | TTGGGAGGCTGAGGTGGGAAGATCCCTT | 1 | 5273-5300 |
| CCTGGCAGTAAGGGATCTTCCCACCTCA | TGAGGTGGGAAGATCCCTTACTGCCAGG | 1 | 5282-5309 |
| CTCAAACTCCTGGCAGTAAGGGATCTTC | GAAGATCCCTTACTGCCAGGAGTTTGAG | 1 | 5290-5317 |
| TAATGTTGGCAAGGCTGGTCTCAAACTC | GAGTTTGAGACCAGCCTGGCCAACATTA | 1 | 5503-5556 |
| TAAAGGATTATAAAATTTAGGTAGTTTT | AAAACTACCTAAATTTTATAATCCTTTA | 1 | 10818 10845 |
| CCAGATGAAGAAATTAAAGGATTATAAA | TTTATAATCCTTTAATTTCTTCATCTGG | 1 | 10832-10859 |
| CAGGTGCTCCAGATGAAGAAATTAAAGG | CCTTTAATTTCTTCATCTGGAGCACCTG | 1 | 10840-10867 |
| AGGGGCAGGTGCTCCAGATGAAGAAATT | AATTTCTTCATCGGAGCACCTGCCCCT | 1 | 10845-10872 |
| GAATAAGTAGGGGCAGGTGCTCCAGAT | ATCTGGAGCACCTGCCCCTACTTATTTC | 1 | 10854-10881 |
| TTACTGCAATCTTCTTGAAATAAGTAGGG | CCCTACTTATTTCAAAGAAGATTGCAGTAA | 1 | 10869-10897 |
| TTTACTGCAATCTTCTTGAAATAAAGTAGG | CCTACTTATTTCAAGAAGATTGCAGTAAA | 1 | 10870-10898 |
| ATGTTCCCTCATTTAATCGTTTTACTGC | GCAGTAAAACGATTAAATGAGGGAACAT | 1 | 10891-10918 |
| CTGCATATGTTCCCTCATTTAATCGTTT | AAACGATTAAATGAGGGAACATATGCAG | 1 | 10897-10924 |
| TCCCTCATTTAATCGTTTTACTGCAATC | GATTGCAGTAAAACGATTAAATGAGGGA | 1 | 10887-10914 |
| AAGGAGACATGACTACTAAGGACACATG | CATGTGTCCTTAGTAGTCATGTCTCCTT | 2 | 11397-11414 |
| GACTACTAAGGACACATGCAGCGTGGTA | TACCACGCTGCATGTGTCCTTAGTAGTC | 2 | 11387-11414 |
| ACTAAGGACACATGCAGCGTGGTATCTT | AAGATACCACGCTGCATGTGTCCTTAGT | 6 | 11383-11410 |
| CCAATTGGCTCAAAAACTAAGAATGATT | AAATCATTCTTAGTTTTTGAGCCAATTGG | 6 | 14306-14333 |
| CAAAAAACTAAGAATGATTTTGACCTTAT | ATAAGGTCAAAAAATCATTCTTAGTTTTTG | 6 | 14296-14323 |
| AAGAATGATTTTGACCTTATAAAAACGT | ACGTTTTTATAAGGTCAAAAAATCATTCTT | 6 | 14288-14315 |
| TTGACCTTATAAAAACGTTGTTTAAAAA | TTTTTAAACAACGTTTTTATAAGGTCAA | 6 | 14278-14305 |
| GTTTAAAAAACAAATATGTAACAGAAAC | GTTTCTGTTACATATTTGTTTTTTAAAC | 6 | 14259-14286 |
| ATGTAACAGAAACCATATGGCCCACAGT | ACTGTGGGCCATATGGTTTCTGTTACAT | 6 | 14244-14271 |
| CTAAAGTATTTATGATTTGACCCCTTAC | GTAAGGGGTCAAATCATAAATACTTTAG | 6 | 14216-14243 |
| TTGACCCCTTACAGAAAAACTGTGGACC | GGTCCACAGTTTTTCTGTAAGGGGTCAAA | 6 | 14200-14227 |
| GAACAGCAGGCCAAATCCAATTGGCTCA | TGAGCCAATTGGATTTGGCCTGCTGTTC | 6 | 14248-14275 |
| GGTAAGCTATGAACAGCAGGCCAAATCC | GGATTTGGCTGCTGTTCATAGCTTACC | 6 | 14332-14349 |
| ACCTATTGGCACCCGAATATATTTGTAG | CTACAAATATATTCGGGTGCCAATAGGT | 6 | 17428-17455 |
| GGCACCCGAATATATTTGTAGAATGAAT | ATTCATTCTACAAATATATTCGGGTGCC | 6 | 17421-17448 |
| TATACCAAGTACCTATTGGCACCCGAAT | ATTCGGGTGCCAATAGGTACTTGGTATA | 6 | 17438-17465 |
| GGGGCCAAAAAACCTTATACCAAGTACCT | AGGTACTTGGTATAAGGTTTTTGGCCCC | 6 | 17452-17479 |

TABLE 3
Guides targeting the ds Regions in stem
in the XIST trascript (total crRNAs = 12)

| | | | TARGET |
|---|---|---|---|
| SENSE | ANTISENSE | EXON | SITE |
| AGCGGTAGGTACA | TTGGTGGTGTGTGA | 1 | 840- |
| CTCACACACCACC | GTGTACCTACCGCT | | 867 |
| AA | | | |
| ATCCGCCATTTTGG | CTTTGTTAGGTTGT | 1 | 1240- |
| ACAACCTAACAAAG | CCAAAATGGCGGAT | | 1267 |
| TGAATTCTACAAAT | TATTAAGAGGCTTT | 1 | 2357- |
| AAAGCCTCTTAATA | TTTTGTAGAATTCA | | 2384 |
| GTGGCCAACACAGT | ATCTTTTCTTGTGT | 1 | 3300- |
| ACACAAGAAAAGAT | ACTGTGTTGGCCAC | | 3327 |
| ACAAATACAATCAC | GCCTCCCAATATGT | 1 | 6010- |
| ACATATTGGGAGGC | GTGATTGTATTTGT | | 6037 |
| CCAGACGATTATAA | CATGTTGTGTGTGA | 1 | 6290- |
| TCACACACAACATG | TTATAATCGTCTGG | | 6317 |
| ACTGATGGCTGAA | TGATTGTCCCATTT | 1 | 10134- |
| AAATGGGACAATC | TTTCAGCCCATCAG | | 10161 |
| A | T | | |
| TTCTATCCACAGAC | ACTGAGGGTGGTGG | 6 | 10695- |
| CCACCACCCTCAGT | GTCTGTGGATAGAA | | 10722 |
| CACTAGAAATCCCA | AGGATTCTGGGGTT | 6 | 13823- |
| AACCCCAGAATCCT | TGGGATTTCTAGTG | | 12850 |
| CAAAATTACCAGAG | ATTTGTGTTTGCTG | 6 | 14659- |
| CAGCAAACACAAAT | CTCTGGTAATTTTG | | 14686 |
| CGGAAAAGGTCAAA | AGGCCTGGCTGGGC | 6 | 18192- |
| GCCCAGCCAGGCCT | TTTGACCTTTTCCG | | 18210 |
| TGACATTTATCTAT | AAGTGGAGAAGGAA | 6 | 18648- |
| TTCCTTCTCCACTT | ATAGATAAATGTCA | | 18675 |

TABLE 4
Guides targeting the ss and ds regions
in stem-loopjunction in the XIST
transcript (Total crRNAs = 22)

| | | | TARGET |
|---|---|---|---|
| SENSE | ANTISENSE | EXON | SITE |
| GAGAGAAGCTGGGC | AGTTCCTCAGTCCC | 1 | 2929- |
| GGGACTGAGGAACT | GCCCAGCTTCTCTC | | 2956 |
| TTTCGAGAGAAGCT | CCTCAGTCCCGCCC | 1 | 2933- |
| GGGCGGGACTGAGG | AGCTTCTCTCGAAA | | 2960 |
| AGTGACTTTCGAGA | TCCCGCCCAGCTTC | 1 | 2969- |
| GAAGCTGGGCGGGA | TCTCGAAAGTCACT | | 2999 |
| CAGGGCAATTGTCT | AAAAAAAAAAAGTA | 1 | 5339- |
| TACTTTTTTTTTTT | AGACAATTGCCCTG | | 5366 |
| AAATTGTCTTACTT | ATTAAAAAAAAAAA | 1 | 5333- |
| TTTTTTTTTTTTAA | AAAGTAAGACAATT | | 5360 |
| T+0 | | | |
| TTACTTTTTTTTTT | GGCCAACATTAAAA | 1 | 5326- |
| TTTTAATGTTGGCC | AAAAAAAAAAGTAA | | 5355 |
| ATATGCTTTTTAAA | TATGCAGAGGTGCT | 1 | 10918- |
| GCACCTCTGCATA | TTTAAAAAGCATAT | | 10945 |
| AAAGGTGGCATATG | GTGCTTTTAAAAAG | 1 | 10927- |
| CTTTTTAAAAGCAC | CATATGCCACCTTT | | 10954 |
| GTGGCATATGCTTT | AGAGGTGCTTTTAA | 1 | 10923- |
| TTAAAAAAGCACCT | AAAAGCATATGCCA | | 10950 |
| CT | C | | |
| GGCATATGCTTTTT | GCAGAGGTGCTTTT | 1 | 1092- |
| AAAAGCACCTCTGC | AAAAAGCATATGCC | | 10948 |
| CTCACATGCTCAGA | CTCCTCTTGGACAT | 2 | 11428- |
| TGTCCAAGAGGAG | TCTGAGCATGTGAG | | 11455 |
| GCTCAGAATGTCCA | CCTTAGGCTCCTCT | 2 | 11421- |
| AGAGGAGCCTAAGG | TGGACATTCTGAGC | | 11448 |
| CAGAATGTCCAAGA | TCTCCTTAGGCTCC | 2 | 11418- |
| GGAGCCTAAGGAGA | TCTTGGACATTCTG | | 11445 |
| GGTCTCACATGCTC | CTCTTGGACATTCT | 2 | 11431- |
| AGAATGTCCAAGAG | GAGCATGTGAGACC | | 11456 |
| GAATAACAAATAAT | CCACCCCCTGATGT | 6 | 14356- |
| ACATCAGGGGGTGG | ATTATTTTTTATTC | | 14385 |
| AATACATCAGGGGG | TTCATAGCTTACCA | 6 | 14347- |
| TGGTAAGCTATGAA | CCCCCTGATGTATT | | 14374 |
| AAATAATACATCAG | TAGCTTACCACCCC | 6 | 14351- |
| GGGGTGGTAAGCTA | CTGATGTATTATTT | | 14378 |
| AATAACAAATAATA | ACCACCCCCTGATG | 6 | 14357- |
| CATCAGGGGGTGGT | TATTATTTGTTATT | | 14384 |
| GGAAGGCATGCATT | AGACATGGGAAAAA | 6 | 17482- |
| TTTTTCCCATGTCT | AATGCATGCCTTCC | | 17509 |
| CTCTGGGAAGGCAT | TGGAAAAAAATGCA | 6 | 17487- |
| GCATTTTTTTCCCA | TGCCTTCCCAGAG | | 17514 |
| GCATGCATTTTTTT | CCCAGAGACATGGG | 6 | 17477- |
| CCCATGTCTCTGGG | AAAAAAATGCATGC | | 17507 |
| CATGCATTTTTTTC | CCCCAGAGACATGG | 6 | 17476- |
| CCATGTCTCTGGGG | GAAAAAAATGCATG | | 17503 |

### Example 3

### (1) Plasmid Construction and Target Guide

To create a PA magnet system, the DNA sequence of dead Lwa (also known as “dCas13a”; from Leptotrichia wadei) was amplified in two parts and fused with pcDNA3.1-PA by using Hifi DNA Assembly (NEB) (which is referred to as “pPA-dCas13-EGFP”). A Lwa-specific scaffold RNA sequence was inserted using Hifi DNA Assembly (NEB). A phosphorylated oligonucleotide encoding an sgRNA sequence was ligated to Bbs1-digested scaffold constructs to prepare sgRNA-targeting XistRNAs and fusion RNAs.

### (2) Confirmation of Target RNA Localization

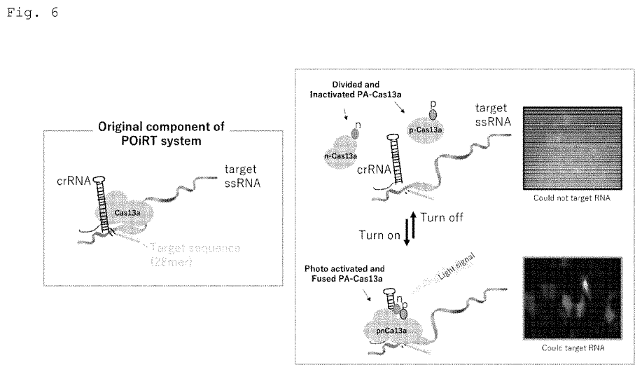

A short-chain-sequence-targeting sgRNA designed based on RNA conformation prediction and pPA-dCas13-EGFP were introduced into the HEK293T cells. The binding to the target XIST RNA was observed under a fluorescence microscope (FIG. 6).

### (3) Evaluation of Target RNA Function: ChIP-qPCR

The binding of the target XIST RNA to an arbitrary sequence on chromatin was confirmed. A short-chain-sequence-targeting sgRNA designed based on RNA conformation prediction and pPA-dCas13-EGFP were introduced into the HEK293T cells. Thereafter, the XIST-sgRNA-Cas13-EGFP complex on chromatin was crosslinked with 1% paraformaldehyde, and the Cas13-EGFP fusion proteins in the cell extract were immunoprecipitated with anti-GFP antibodies. Thereafter, the co-immunoprecipitated XIST and XIST-binding genomic sequences were extracted. The extracted genomic sequences were detected by using the qPCR method to confirm the polymerization of XIST-chromatin. Histone was used as a positive control, while non-specific IgG was used as a negative control (FIG. 7).

### Example 4

### (1) Plasmid Construction and Target Guide

To create an RNA-editing system, the EGFP domain of pPA-dCas13-EGFP was re-written into a domain in which APOBEC1 domain and A1CF domain were fused using Hifi DNA Assembly (NEB) (referred to as “RESCUE system”; pPA-dCas13-ABC1A1, FIG. 8). A phosphorylated oligonucleotide encoding an sgRNA sequence was ligated to Bbs1-digested scaffold constructs to prepare an RNA fused with sgRNA-targeting RNA of APP protein with Aβ cleavage sequence recognition.

### (2) Testing of Target RNA Gene Editing Effect

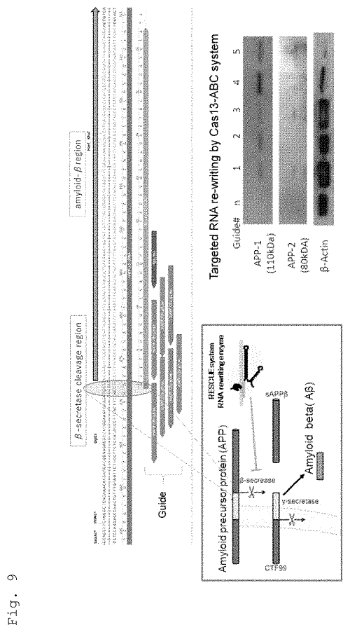

The function of the target APP protein, a short-chain-sequence-targeting sgRNA designed based on RNA conformation prediction, and pPA-dCas13-ABC1A1 were introduced into the HEK293T cells. The effect of inhibiting cleavage by β-secretase on the target APP protein was confirmed by western blot analysis (FIG. 9).

1-13. (canceled)

14. A nucleic acid construct comprising at least one guide RNA portion that binds to one or more target RNAs and an RNA-cleaving Cas protein expression portion, wherein
the at least one guide RNA binds to a single-stranded region (ss region) of the one or more target RNAs, and
the one or more target RNAs contain a mutation associated with canceration of a vertebrate cell, and are expressed in a cancer cell and not produced in a normal cell, provided that the mutation excludes single-nucleotide polymorphisms and mutation of introns.

15. The nucleic acid construct according to claim 14, wherein an RNA-cleaving Cas protein is a Cas13 family protein.

16. The nucleic acid construct according to claim 15, wherein the RNA-cleaving Cas protein is C2C2.

17. The nucleic acid construct according to claim 14, wherein at least one guide RNA targets RNA that contains a mutation associated with canceration of a vertebrate cell.

18. The nucleic acid construct according to claim 14, wherein
the mutation in a vertebrate cell is a translocation, and
at least one guide RNA targets RNA that corresponds to a gene of the translocation.

19. A pharmaceutical composition comprising the nucleic acid construct of claim 14 as an active ingredient.

20. An anticancer agent comprising a nucleic acid construct as an active ingredient,
the nucleic acid construct comprising at least one guide RNA portion that binds to one or more target RNAs and an RNA-cleaving Cas protein expression portion, wherein
at least one guide RNA binds to a single-stranded region (ss region) of the one or more target RNAs, and
the one or more target RNAs contain a mutation associated with canceration of a vertebrate cell, and are expressed in a cancer cell and not produced in a normal cell, provided that the mutation excludes single-nucleotide polymorphisms and mutation of introns.

21. The nucleic acid construct according to claim 14, wherein the one or more target RNAs correspond to one or more translocations.

22. A nucleic acid construct comprising at least one guide RNA portion that binds to one or more target RNAs, an RNA-non-cleaving Cas protein expression portion, an RNA-editing enzyme portion, and a coenzyme portion for an RNA-editing enzyme, wherein the one or more target RNAs contain a sequence that is a target for editing.

23. The nucleic acid construct according to claim 14, wherein
the RNA-non-cleaving Cas protein is composed of two components, the two components forming a dimer by light irradiation, thus forming an active RNA-non-cleaving Cas protein.

24. The nucleic acid construct according to claim 14, wherein the RNA-cleaving Cas protein is capable of indiscriminately reducing expression of mRNA in a vertebrate cell.

25. The nucleic acid construct according to claim 14, wherein the RNA-cleaving Cas protein is C2C2 derived from Leptotrichia shahii.

26. The anticancer agent according to claim 20, wherein the RNA-cleaving Cas protein is capable of indiscriminately reducing expression of mRNA in a vertebrate cell.

27. The anticancer agent according to claim 20, wherein the RNA-cleaving Cas protein is C2C2.

28. The nucleic acid construct according to claim 15, wherein at least one guide RNA targets RNA that contains a mutation associated with canceration of a vertebrate cell.

29. The nucleic acid construct according to claim 15, wherein
the mutation in a vertebrate cell is a translocation, and
at least one guide RNA targets RNA that corresponds to a gene of the translocation.

30. A pharmaceutical composition comprising the nucleic acid construct of claim 15 as an active ingredient.

31. A pharmaceutical composition comprising the nucleic acid construct of claim 18 as an active ingredient.

32. A pharmaceutical composition comprising the nucleic acid construct of claim 29 as an active ingredient.
